(19) United States
(12) Patent Application Publication
(10) Pub. No.: US 2005/0038370 A1
(43) Pub. Date: Feb. 17, 2005

(54) Tissue anchor for endorobots

(76) Inventors: Rainer Kuth, Herzogenaurach (DE); Martin Requardt, Nuernberg (DE)

Correspondence Address: HARNESS, DICKEY & PIERCE, P.L.C., P.O.BOX 8910, RESTON, VA, 20195, US

(21) Appl. No.: 10/915,382

(22) Filed: Aug. 11, 2004

(30) Foreign Application Priority Data
Aug. 11, 2003 (DE) 10336734.9

(51) Int. Cl. A61F 13/00

(52) U.S. Cl. 602/78

(57) ABSTRACT

A fastening device is for anchoring a surgical or diagnostic medical aid in the tissue of a human or animal hollow organ. The fastening device includes an anchor head, which is configured to penetrate the tissue; a driving device for driving the anchor head into the tissue; and a trigger device to trigger the driving of the anchor head into the tissue.

The present application hereby claims priority under 35 U.S.C. §119 on German patent application number DE 103 36 734.9 filed Aug. 11, 2003, the entire contents of which are hereby incorporated herein by reference.

### FIELD OF THE INVENTION

The invention generally relates to a fastening device for a probe inside a human or animal body.

### BACKGROUND OF THE INVENTION

An endoscope is used to examine the upper and lower gastrointestinal tract, which records individual images of the surroundings and transmits them to an external image processor. Lesions such as tumors can be identified and located on the basis of such images.

In addition to examinations undertaken in the gastrointestinal tract, endoscopy is also used in other hollow organs and cavities in the human or animal body. Examples of this include examinations of blood vessels, the abdominal cavity—which is typically undertaken by means of a small incision to the navel—or an examination of the lungs.

Cordless or wireless endoscopic probes are currently used for the care of patients undergoing endoscopic diagnosis. These are capsules, which include specific devices such as an image recorder with a transmitter for transmitting the recorded image data. To aid navigation, a magnet can often be found in such capsules, also referred to as endorobots, which in turn enables control by way of an external magnetic field.

The capsule endoscope or endorobot is preferably inserted into the gastrointestinal tract orally or anally. In hollow organs or cavities in the body that are closed off externally, the endorobot can be inserted through a small incision.

Although lesions can be successfully identified and located with the aid of endoscopy, it still remains difficult for a surgeon to relocate the identified position during subsequent examinations or interventions. Since a human intestine can reach up to 11 m in length, and has no landmarks and is constantly moving, it is extremely difficult for a surgeon to relocate a previously identified lesion, i.e. during preparation for an operation.

In principle, it is possible to mark a lesion by way of chromoendoscopy and intravital staining. With the techniques, a colored solution is applied to the mucosa of the gastrointestinal tract, causing specific discoloration of mucosa modified by disease. The intracoporal position of the marking thus applied however fails to permit extracorporal location of the marked position.

For location purposes, cordless probes can be guided to the previously identified or marked position, the position of the probes inside the body being easily located or detected from the outside. The probes used are generally the endorobots, which in some instances were used for prior identification and possibly also the marking of the lesion, i.e. as endomarkers.

One disadvantage here is that after positioning, in particular during the time between diagnosis and operation, the probes can change their position. For example, in blood vessels, the probes move with the flow of blood. In the gastrointestinal tract, both the movement of the organ itself and also the substances transported therein result in the probe moving position over time.

Since at least one hour generally passes between the diagnosis and subsequent operation, it is a common occurrence for the probe to have moved from the originally marked position by the start of the operation. The practical benefit of a corresponding use of surgical or diagnostic aids, such as for example probes or endorobots, is thus reduced.

### SUMMARY OF THE INVENTION

An object of an embodiment of the present invention is thus to provide a way/device to prevent the displacement of an initially positioned surgical or diagnostic medical aid, such as a probe.

An object may be achieved by the use of a fastening device.

According to an embodiment of the present invention, a fastening device is proposed for anchoring a surgical or diagnostic medical aid in the tissue of a human or animal hollow organ. The fastening device includes an anchor head, which is configured to penetrate the tissue, a driving device for driving the anchor head into the tissue and a trigger device for triggering the driving of the anchor head into the tissue.

With an inventive fastening device, a surgical or diagnostic medical aid can be securely anchored in a previously identified position, in order to prevent subsequent displacement of the aid in a reliable manner.

In order to prevent the driving force acting on the anchor head before the fastening device is triggered, the trigger device is expediently integrated in the driving device.

Advantageous multiple use can be achieved by using a spring element as the driver in the driving device. Alternatively, the driving device can include a gas pressure element as the driver, thereby achieving a large driving force with a small structure.

The driving unit is preferably equipped with a runner to hold the anchor head to ensure effective transmission of the driving force to the anchor head.

At least one anchoring element is configured on the anchor head to prevent the anchor head detaching from the tissue after penetration. This is expediently in the form of a barb. Maintaining a small cross-sectional area of the anchor head facilitates the penetration of the anchor head into the tissue. In addition, the at least one anchoring element is expediently configured as an expansion device with at least one arbor, whereby the arbor can be opened out by way of an opening device, or can be configured as a self-expanding arbor, when subject to tensile force.

### BRIEF DESCRIPTION OF THE DRAWINGS

Exemplary embodiments of the invention will be explained in more detail below with reference to the drawings, in which:

FIG. 1a shows an endorobot with an inventive fastening device in standby position

FIG. 1b shows the endorobot in FIG. 1a anchored to a tissue

FIG. 2 shows a first embodiment of an inventive fastening device

FIG. 3 shows a second embodiment of an inventive fastening device and,

FIG. 4 details the different embodiments of an anchor head according to the present invention.

### DETAILED DESCRIPTION OF THE PREFERRED EMBODIMENTS

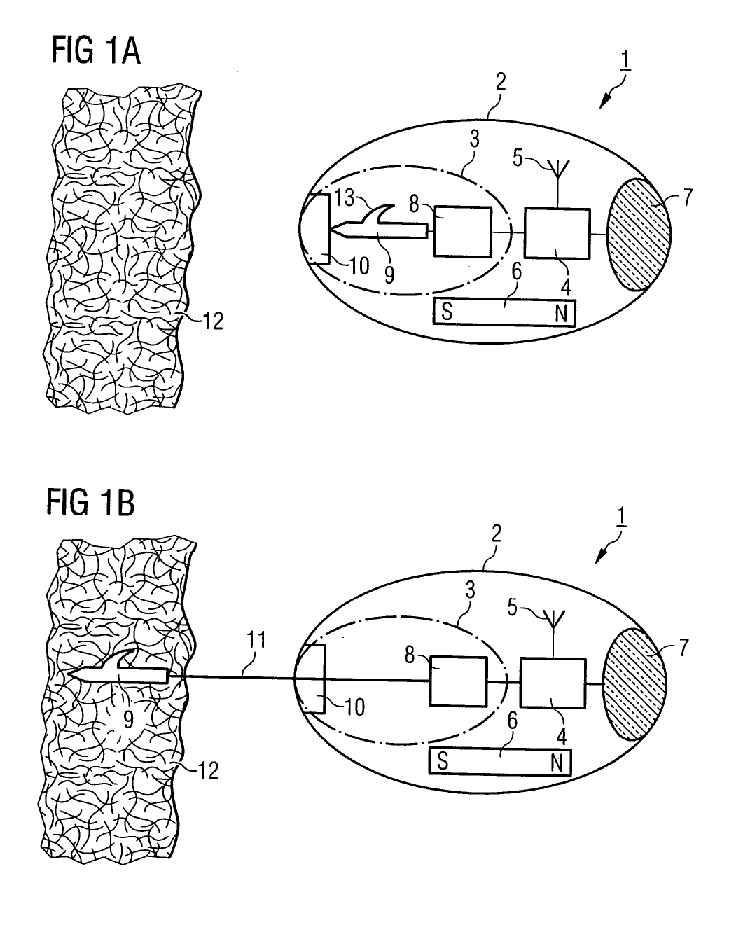

FIG. 1 shows the endorobot 1 configured as a wireless endoscope. The endorobot 1 is in the form of a consumable capsule; its shell 2 is manufactured from a biocompatible material which is resistant to the digestive secretions occurring in the gastrointestinal tract. An inventive fastening device 3 is arranged within the capsule shell 2 adjacent to one end.

Other functional components of the endorobot 1 are housed in the remaining space within the capsule 2. Typically these devices include an optical mapping system 7 for producing images of the surroundings of the endorobot 1. A central electronic signal processor 4 transforms optical image signals to electrical image signals and controls the endorobot. It is equipped with an antennae device 5 for wireless communication with an external signal processor. A permanent magnet 6 within the capsule shell enables the orientation or guiding of the endoscopic probe 1 from outside a human or animal body.

The inventive fastening device 3 includes three basic components, namely a driving device 8, an anchor head 9, and a trigger device 10. The trigger device 10 can be controlled by way of electronic signal processor 4. In the schematic representation in FIG. 1, the trigger device 10 is configured as a blockade device for the exit of the anchor head 9 from the capsule shell 2.

Alternatively the trigger device can also be integrated in the driving device 8 in such a way that the driving force developed therein only acts on the anchor head 9 after the trigger device 10 has been triggered. The end of the anchor head 9 to be sunk into the tissue in this instance extends to or close to the one end of the capsule shell 2. To avoid contamination of the interior of the endorobot, this one end can additionally be equipped with a sealing mechanism, which opens synchronously with the trigger device 10. Instead of the sealing mechanism, the wall of the capsule shell can also be configured to be so thin at this point that it can be penetrated when the anchor head 9 is driven forward.

To fasten the endorobot 1 to a previously determined position in the body of a patient, a corresponding signal is transmitted by a wireless communication to the signal processor 4. This activates the trigger device 10 and thus enables the driving force stored in the driving device 8 to act on the anchor head 9. As a result of the driving force acting on it, the anchor head 9 moves at high speed towards the tissue wall 12 facing it and penetrates its surface.

To prevent the anchor head 9 becoming detached from the endorobot 1, it is preferably connected by way of a flexible connection 11, for example a cord or a flex or similar, to part of the endorobot 1, for example the capsule shell 2 or a device within the endorobot 1. In order that the action of a tensile force on the anchor head 9 does not cause said anchor head 9 to detach from the tissue wall 12, an anchoring element 13 is configured thereon.

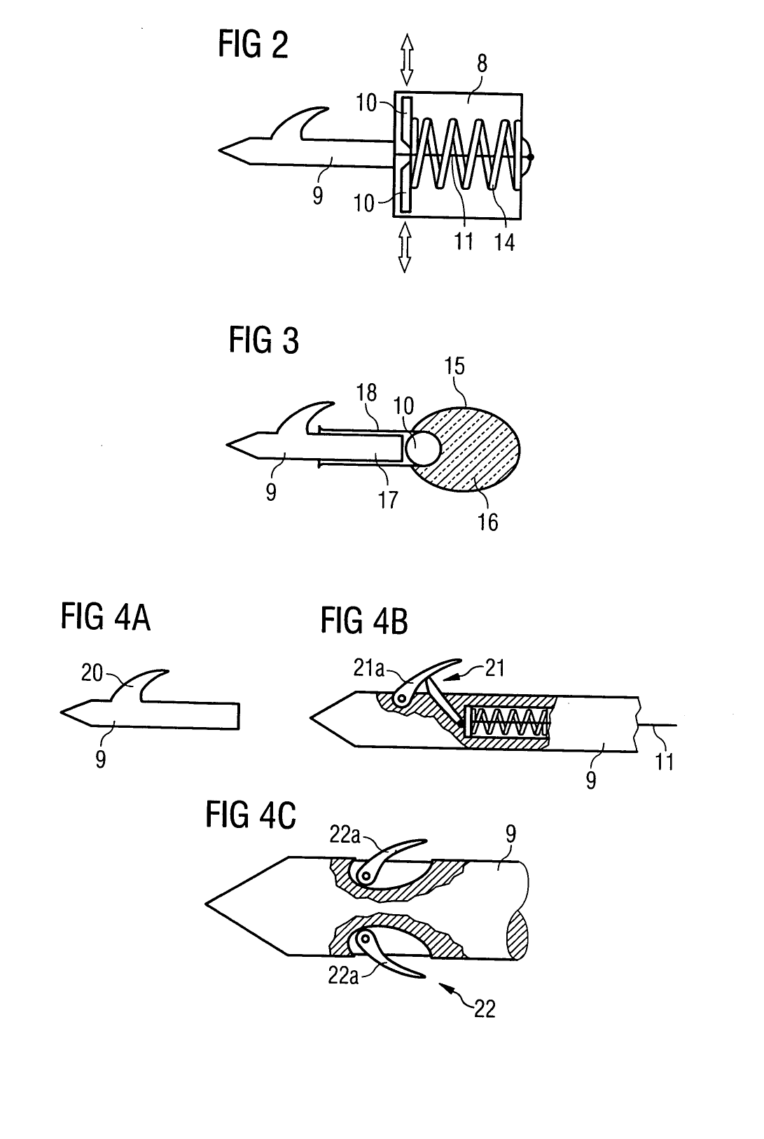

In the simplest case as shown in FIG. 2, the driving device 8 is configured in the form of a spring element 14, for example a spiral spring. In the initial state the spring element 14 is tensioned. The tension is maintained by the trigger device 10 in its closed state and cannot be transmitted to the anchor head 9. When the trigger device 10 opens, the spring element 14 can become slack, whereby the energy thereby released is transmitted to the anchor head and ejects this from the capsule shell 2 of the endorobot 1.

In an alternative embodiment, which is shown in FIG. 3, the driving force is provided by a pressurized gas 16 in a pressure vessel 15. The trigger device 10 seals off the gas chamber of the pressure vessel 15 from the outside. When triggered, the trigger device 10 releases the opening of the pressure vessel 15, so that the gas pressure acts directly on the anchor head 9 and ejects the anchor head from the endorobot 1. For this purpose, the anchor head preferably has a shank-like segment 17, which is arranged in the runner 18 connected to the pressure vessel in the standby position.

FIG. 3 shows several preferred embodiments of an anchor head 9 for use in an inventive fastening device 3. In order to prevent the anchor head 9 being drawn out of the tissue wall 12 when subject to a tensile force, one or more anchoring elements 13 are arranged adjacent to its front free end, which is preferably configured as a point 19. The anchoring elements 13 can be fixed e.g. in the form of a barb, or movable, e.g. such as expansion devices 21 or 22.

The anchoring element 13 is expediently configured as an arbor in the form of a barb 20, as shown in the anchor head detail in FIG. 3a. The front edge of the barb 20 preferably tapers to a point so that no appreciable resistance counteracts the penetration of the anchor head 9 into the tissue. The rear flank of the barb 20 is preferably configured as level so that the barb abuts firmly against the tissue when subject to tensile loading.

To keep the level of work required for the anchor head 9 to enter a tissue wall 12 as low as possible, the anchoring element 13 can also be configured in the form of an expansion device 21 or 22 as shown in FIGS. 3b and 3c. In standby mode, i.e. before the fastening device 3 is triggered, an arbor 21a or 22a of the expansion device 21 or 22 is disposed on the shank-like part of the anchor head. This is the rest position of the expansion device. In the tissue wall, the moveable arbor disposed on the anchor head 9 expands away from this to enable it to grip the tissue in the same way as a barb.

In a first embodiment 21 of the expansion device, the arbors 21a are opened out by way of an opening device 23 from their rest position, in order to form an open, acute angle in relation to the rear end of the anchor head 9. The opening device 23 preferably only opens the arbor 21a out after penetration of the anchor head into the tissue in order to keep its penetration resistance to a minimum. This can be achieved by triggering the opening device by means of a tensile stress on the flexible connection 11.

The alternative embodiment 22 of an expansion device shown in FIG. 3c includes one or several arbors 22a, which are each arranged in a rotatable manner in a cavity in the anchor head 9. The pointed ends of the arbors oriented towards the rear end of the anchor head however protrude somewhat out of the cavity. During penetration of the anchor head 9 into a tissue wall 12, they rest against this. If tensile force is exerted on the anchor head 9, the pointed ends of the arbors push into the surrounding tissue and stand up. The anchor head is thereby securely anchored in the tissue.

The effectiveness of the inventive fastening device 3 for endorobots 1 is not limited to penetration by the anchor head. Anchoring is also effective in the case of thin tissue walls, which are penetrated by the anchor head 9 during the fastening process. The barb 20 or one of the expansion devices 21 or 22 successfully prevent withdrawal of the anchor head 9 from the tissue wall here too.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. Fastening device for anchoring at least one of a surgical and diagnostic medical aid in tissue of at least one of a human and animal hollow organ, the device comprising:
an anchor head, configured to penetrate the tissue;
a driving device for driving the anchor head into the tissue; and
a trigger device for releasing the driving of the anchor head into the tissue.

2. Fastening device according to claim 1, wherein the trigger device is integrated in the driving device.

3. Fastening device according to claim 1, wherein the driving device includes a spring element as a driver.

4. Fastening device according to claim 1, wherein the driving device includes a gas pressure element as a driver.

5. Fastening device according to claim 1, wherein the driving device includes a runner to hold the anchor head.

6. Fastening device according to claim 1, wherein the anchor head includes a shank-like segment.

7. Fastening device according to claim 1, wherein at least one anchoring element is configured on the anchor head.

8. Fastening device according to claim 7, wherein the at least one anchoring element has the form of a barb.

9. Fastening device according to claim 7, wherein the at least one anchoring element is configured as an expansion device with at least one arbor.

10. Fastening device according to claim 9, wherein the at least one arbor of the expansion device is openable by an opening device.

11. Fastening device according to claim 9, wherein the at least one arbor of the expansion device is configured as a self-expanding arbor when subject to a tensile force.

12. Fastening device according to claim 2, wherein the driving device includes a spring element as a driver.

13. Fastening device according to claim 2, wherein the driving device includes a gas pressure element as a driver.

14. Fastening device according to claim 2, wherein the driving device includes a runner to hold the anchor head.

15. Fastening device according to claim 2, wherein the anchor head includes a shank-like segment.

16. Fastening device according to claim 5, wherein the anchor head includes a shank-like segment.

17. Fastening device according to claim 2, wherein at least one anchoring element is configured on the anchor head.

18. Fastening device according to claim 17, wherein the at least one anchoring element has the form of a barb.

19. Fastening device according to claim 8, wherein the at least one anchoring element is configured as an expansion device with at least one arbor.

20. Fastening device according to claim 10, wherein the at least one arbor of the expansion device is configured as a self-expanding arbor when subject to a tensile force.

21. A device for anchoring a probe in tissue of at least one of a human and animal organ, the device comprising:
means for penetrating the tissue;
means for driving the anchor head into the tissue; and
means for releasing the driving of the anchor head into the tissue.

22. The device according to claim 21, wherein the means for driving and the means for releasing are integrated.

23. The device according to claim 21, wherein the means for penetrating includes an anchor head.

24. The device according to claim 23, wherein the anchor head includes a shank-like segment.

25. The device according to claim 23, wherein at least one anchoring element is configured on the anchor head.

26. The device according to claim 21, wherein the medical aid is at least one of a surgical and diagnostic medical aid.

27. The device according to claim 21, wherein the fastening device is for anchoring a probe in tissue of at least one of a human and animal hollow organ.

28. A medical aid including the fastening device of claim 1.

29. A probe including the fastening device of claim 1.

30. A probe including the device of claim 21.
